(19) United States
(12) Patent Application Publication
(10) Pub. No.: US 2012/0177631 A1
(43) Pub. Date: Jul. 12, 2012

(54) Composition for Health Promoting Compounds

(76) Inventors: Morteza Naghavi, Houston, TX (US); Albert Andrew Yen, Pearland, TX (US); Mortaza Mark Naghavi, Katy, TX (US)

(21) Appl. No.: 13/346,811

(22) Filed: Jan. 10, 2012

Related U.S. Application Data
(60) Provisional application No. 61/460,905, filed on Jan. 10, 2011.

(51) Int. Cl. A61K 33/30; A61P 3/00; A61P 3/06; A61K 36/899; A61P 9/00

(52) U.S. Cl. 424/115; 424/643

(57) ABSTRACT

This invention relates to orally ingested compositions for improving cardiovascular health and reducing the overall levels of cholesterol by lowering the levels of low density lipoprotein (LDL) and triglycerides, increasing the level and density of high density lipoprotein (HDL), assisting with blood pressure control, or improve some aspect of cardiovascular function, and thus preferably assisting with cardiovascular health when administered to mammals, including humans.

### RELATED APPLICATION

This application claims priority from and incorporates herein by reference Provisional Application Ser. No. 61/460,905 entitled Compositions for Health Promoting Compounds filed Jan. 10, 2011.

### BACKGROUND OF INVENTION

This disclosure relates to compositions for enhancing cardiovascular health. The orally ingested compositions contain effective amounts of vitamins, minerals, herbs, and other natural extracts and do not contain dangerous stimulants. Other compositions contain additional compounds which reduce or control levels of cholesterol and triglycerides.

Prior formulations fall short of the unique combinations of this disclosure which include components, such as fish oil, plant sterols and niacin and a statin in an all-in-one formula to promote cardiovascular health.

### DETAILED DESCRIPTION OF THE INVENTION

The compositions described herein are unique combinations of specific vitamins, minerals, herbs, and nutraceutical compounds. Nutraceutical compounds are a food or food product that reportedly provides health and medical benefits, including the prevention and treatment of disease. These essential components protect cardiovascular health and promote heart healthy lifestyle. Other components reduce or control the levels of total cholesterol by lowering low density lipoproteins (LDL), and triglycerides, and may also increase the level and density of high density lipoproteins (HDL).

The composition described in this disclosure may be consumed as a liquid drink. It may be found advantageous to consume the composition as a smoothie drink or other blended liquid. It may also be consumed as an additive to other drinks such as coffee, tea or a soft drink. The composition (multi-component drink) may be carbonated.

Other components of this disclosure are unique combinations of pharmaceuticals and nutraceuticals. These unique blends of elements are called polyceuticals. Examples of polyceuticals are: a combination of aspirin and vitamin B6; a combination of aspirin and vitamin B12; a combination of aspirin and folic acid; a combination of ibuprofen and COQ10; a combination of a nutraceutical composition for boosting metabolism and a pharmaceutical medication for suppressing appetite, such as phentermine; a combination of a nutraceutical composition for increasing levels of high density lipoprotein (HDL, also referred to as “good” cholesterol) and a pharmaceutical medication for reducing levels of low density lipoprotein (LDL, also referred to as “bad” cholesterol), such as medication belonging to the statin class of medications.

One embodiment comprises a composition containing folic acid, magnesium taurate, niacin, Quercetin, resveratrol, vitamin B6, vitamin B12, vitamin D, and zinc. In order to secure the desired result of cardiovascular health, the following components are provided:

Folic acid: is used to treat or prevent folic acid deficiency. It is a B-complex vitamin needed by the body to manufacture red blood cells. Folic Acid can be used to help prevent or treat heart disease, Alzheimer's disease, depression, anemia, and certain types of cancer (Refsum H et al., Annual Review of Medicine 49 [1]: 31-62)

Magnesium taurate: Magnesium taurate is a combination of the mineral magnesium and the amino acid derivative taurine (2-aminoethanesulfonic acid). Magnesium taurate is one of the most highly-absorbed forms of magnesium. Magnesium is a mineral needed by every cell of body to help maintain normal cardiovascular, muscle, nerve, bone, and cellular function. It is imperative for heart health and normal blood pressure. Taurine also plays a role in the metabolism of calcium and may affect entry of calcium into heart muscle cells where it is essential in the generation and transmission of nerve impulses. Research shows that taurine prevents arrhythmogenesis by limiting cardiac hypertrophy and calcium overload of the myocardium. Taurine also protects the heart against reperfusion-induced arrhythmias via its properties as a membrane stabilizer and as an oxygen free radical scavenger. (Chahine R et al., Arzneimittelforschung 48 [4]:360-4, PMID: 9608877).

Niacin: Niacin or nicotinic acid, an important B vitamin that may raise HDL, also known as “good” cholesterol, by 15 to 35 percent, while also lowering LDL and triglyceride levels. Niacin can make the skin redden and feel warm to the touch (“flushing”), but this flushing is not harmful. In one embodiment, niacin is included in the form of niacinamide.

Quercetin: is used for treating conditions of the heart and blood vessels including “hardening of the arteries” (atherosclerosis), high cholesterol, heart disease, and circulation problems.

Resveratrol: is a phytoalexin that activates the SiRT1 gene and reportedly has anti-obesity, anti-inflammatory, anti-aging, and anti-cancer properties. Trans-resveratrol is the bioactive form of resveratrol. In one embodiment, the resveratrol contains a minimum of 50% trans-Resveratrol.

Vitamin B6: Vitamin B6 performs a wide variety of functions in human body and is essential for maintaining a good health. For example, vitamin B6 is needed for more than 100 enzymes involved in protein metabolism. The nervous and immune systems need vitamin B6 to function efficiently, and it is also needed for the conversion of tryptophan (an amino acid) to niacin. Vitamin B6 also helps maintain blood glucose within a normal range. When caloric intake is low, the human body needs vitamin B6 to help convert stored carbohydrate or other nutrients to glucose to maintain normal blood sugar levels. It is also essential in production of hemoglobin and transfer of oxygen carried by hemoglobin. In one embodiment, the vitamin B6 may be in the form of Pyridoxine HCl.

Vitamin B12: a vitamin, 100 to 250 mcg (microgram), an essential nutrient that speeds metabolism and boosts energy level. In one embodiment, the vitamin B12 is in the form of cyanocobalamin.

Vitamin D: is the precursor molecule to cholesterol. Low vitamin D levels were associated with cardiovascular death, as well as with obesity, hypertension, glucose intolerance, and metabolic syndrome. In one embodiment, vitamin D may be in the form of Cholecalciferol.

Phytosterols, also called plant sterols, are phytochemicals naturally occurring in plants. Phytosterols have cholesterol-lowering properties, by reducing cholesterol absorption in intestines (Ostlund et al., Am J Clin Nutr, 2003; 77:1385-1589).

Zinc: Zinc plays a critical role in protein synthesis and carbohydrate metabolism. More than 300 enzymes in the human body require zinc to function properly. Zinc is vital to the immune system, reproduction, digestion, cell growth, taste, smell, memory, healing as well as reduce inflammation. Zinc also possesses antioxidant properties, protecting the body from free radicals. In one embodiment, zinc is supplied in the form of zinc sulfate. Zinc sulfate supplementation helps to promote a healthy immune system; it is vital in the production of T-cells, which help fight infections.

Another composition also contains folic acid, magnesium taurate, niacin, Quercetin, resveratrol, vitamin B6, vitamin B12, vitamin D, and zinc, but is further comprised of plant sterols (phytosterols) and fish oil—Omega-3. In order to secure the desired result of cardiovascular health, the following components are provided:

Plant sterol (phytosterols): Phytosterols, also called plant sterols are phytochemicals naturally occurring in plants. Phytosterols have cholesterol-lowering properties, by reducing cholesterol absorption in intestines (Ostlund et al., Am J Clin Nutr, 2003; 77:1385-1589).

Omega-3 fish oil: a class of essential polyunsaturated fatty acids found in high concentrations in salmon, halibut, sardines, albacore, and other fatty fishes, has been shown to lower total cholesterol and triglyceride levels, when ingested. Moreover, daily ingestion of omega-3-fatty acids has been linked to prevention of sudden cardiac death in patients who recently survived a heart attack (GISSI-Prevention trial, Circulation, 2002; 105:1897-1903).

Another embodiment of the disclosure comprises a composition also containing folic acid, magnesium taurate, niacin, quercetin, resveratrol, vitamin B6, vitamin B12, vitamin D, and zinc, but is further comprised of L-arginine and plant sterols (phytosterols). Quercetin is a flavonol, is a plant-derived flavonoid found in fruits, vegetables, leaves and grains. It also may be used as an ingredient in supplements, beverages or foods. In order to secure the desired result of cardiovascular health, the following component is provided:

L-Arginine: is used to make the nitric oxide, a compound in the body that relaxes blood vessels. Preliminary studies have found that L-arginine may help with conditions that improve when blood vessels are relaxed (called vasodilatation), such as atherosclerosis, erectile dysfunction, and intermittent claudication.

Another composition subject of this disclosure also contains folic acid, magnesium taurate, niacin, Quercetin, resveratrol, vitamin B6, vitamin B12, vitamin D, and zinc, but is further comprised of L-arginine, and fish oil—Omega-3

Another composition subject of this disclosure also contains folic acid, magnesium taurate, niacin, Quercetin, resveratrol, vitamin B6, vitamin B12, vitamin D, and zinc, but is further comprised of plant sterols (phytosterols), fish oil—Omega-3, and L-arginine

Another composition also contains folic acid, magnesium taurate, niacin, Quercetin, resveratrol, vitamin B6, vitamin B12, vitamin D, zinc, plant sterols (phytosterols), fish oil—Omega-3and and L-arginine, but is further comprised of red yeast. In order to secure the desired result of cardiovascular health, the following component is provided:

Red yeast rice: rice that has been fermented by the red yeast, Monascus purpureus, has been shown to lower blood lipids, including cholesterol and triglycerides, because it contains a substance that inhibits HMG-CoA reductase, the enzyme that is important for the production of cholesterol in the body

Another composition of this disclosure also contains folic acid, magnesium taurate, niacin, Quercetin, resveratrol, vitamin B6, vitamin B12, vitamin D, zinc, plant sterols (phytosterols), fish oil—Omega-3 and L-arginine, but is further comprised of Statin (low dose—less than 20 mg). The nutraceutical composition, in this embodiment, can be referred to as a “Statin Aid.” In order to secure the desired result of cardiovascular health, the following component is provided:

Statins: statins are a class of drugs used to lower cholesterol levels. Increased cholesterol levels are associated with cardiovascular diseases, and therefore, statins are used in the prevention of these diseases.

Another composition subject of this disclosure also contains folic acid, magnesium taurate, niacin, Quercetin, resveratrol, vitamin B6, vitamin B12, vitamin D, zinc, plant sterols (phytosterols), fish oil—Omega-3 and L-arginine, but is further comprised of ubiquinol. The natural ubiquinol form of coenzyme Q10 is 2,3-dimethoxy-5-methyl-6-poly prenyl-1,4-benzoquinol, where the polyprenylated side chain is 9-10 units long in mammals. In order to secure the desired result of cardiovascular health, the following component is provided:

Ubiquinol: is an electron-rich form of coenzyme Q10. It is a lipid-soluble benzoquinol that is found in all cellular systems and in nearly every cell, tissue and organ in mammals. Ubiquinol has an established role as an essential component of the electron transport chain transferring electrons resulting in ATP synthesis. In mammals ATP production takes place predominantly in mitochondria and to a lesser extent in other organelles such as the Golgi apparatus or endoplasmic reticulum. The mitochondria typically produce nearly 95% of the energy required for cellular growth, development and healthy metabolism. The antioxidant action of ubiquinol is considered to be one of the most important functions in cellular systems. Ubiquinol is a potent lipophilic antioxidant capable of regenerating other antioxidants such as tocopherol (Vitamin E) and ascorbate (Vitamin C).

Another composition also contains folic acid, magnesium taurate, niacin, Quercetin, resveratrol, vitamin B6, vitamin B12, vitamin D, zinc, plant sterols (phytosterols), fish oil—Omega-3 and L-arginine, but is further comprised of lycopene. In order to secure the desired result of cardiovascular health, the following component is provided:

Lycopene: as a powerful antioxidant, lycopene helps neutralize harmful free radicals, which are implicated in cancer, heart disease, macular degeneration and other age-related illnesses. The evidence for a benefit is strongest for cancers of prostate, lung, and stomach. Data are also suggestive of a benefit for cancers of pancreas, colon and rectum, esophagus, oral cavity, breast, and cervix.

Another composition also contains folic acid, magnesium taurate, niacin, Quercetin, resveratrol, vitamin B6, vitamin B12, vitamin D, zinc, plant sterols (phytosterols), fish oil—Omega-3 and L-arginine, but is further comprised of β-carotene. In order to secure the desired result of cardiovascular health, the following component is provided:

β-carotene: is a micronutrient which has powerful antioxidant properties. Studies conducted over several years indicate that β-carotene may play a significant role in reducing the effects of certain illnesses such as heart disease, decreased immune function, cataracts, and forms of cancer, and in certain cases, perhaps even prevent them.

Liquid—Solid Combination. In an embodiment, the compositions described herein, which contain only liquid components, are packaged together but not intermixed with other compositions that are contained in solid forms, such as tablets, capsules, or caplets. There are various nutraceutical and pharmaceutical compounds that cannot be chemically combined or physically mixed with each other due to factors such as instability of ingredients, insolubility, viscosity, incompatibility, taste, and reduced shelf life. For example, oil-based liquid products such as fish oil will not mix well with water-based liquids. However, it is often desirable to ingest these compounds at or close to the same time. Therefore, there is a need for a means of enabling a person to store or carry these chemically- or physically- incompatible compounds together so that they may be conveniently ingested together when so desired.

“Polyceutical” Combination. In an embodiment, the compositions described herein, which contain only nutraceutical components, are packaged with pharmaceutical compositions. The inventors have coined the term “Polyceutical” to name the combination of one or more nutraceutical compounds with one or more pharmaceutical compounds, i.e., defined as any chemical substance intended for use in the medical diagnosis, cure, treatment, or prevention of disease.

Examples of polyceuticals are: a combination of vitamin B6 and aspirin; a combination of vitamin B12 and aspirin; a combination of folic acid and aspirin; a combination of CoQ10 and ibuprofen; a combination of one of the nutraceutical compositions described in this disclosure and metformin (an oral antidiabetic drug in the biguanide class); and a combination of one of the nutraceutical compositions described in this disclosure and exenatide (a medication approved for the treatment of diabetes mellitus type 2 belonging to the group of incretin mimetics and administered as a subcutaneous injection).

A polyceutical combination is an effective means of providing two or more compounds that have complementary effects. For example, a polyceutical combination for assisting weight loss can combine a nutraceutical composition that boosts metabolism with a pharmaceutical compound that suppresses appetite, such as the FDA-approved prescription weight loss medication, phentermine. Another example, a polyceutical combination for improving cardiovascular health, can combine a nutraceutical composition that raises HDL (“good”) cholesterol with a statin medication, e.g., atorvastatin, which lowers LDL (“bad”) cholesterol.

This specification is to be construed as illustrative only and is for the purpose of teaching those skilled in the art the manner of carrying out the invention. It is to be understood that the forms of the invention herein shown and described are to be taken as the presently preferred embodiments. As already stated, various changes may be made in the shape, size and arrangement of components or adjustments made in the steps of the method without departing from the scope of this invention. For example, equivalent elements may be substituted for those illustrated and described herein and certain features of the invention maybe utilized independently of the use of other features, all as would be apparent to one skilled in the art after having the benefit of this description of the invention.

While specific embodiments have been illustrated and described, numerous modifications are possible without departing from the spirit of the invention, and the scope of protection is only limited by the scope of the accompanying claims.

What is claimed is:

1. A cardiovascular health-promoting nutraceutical composition comprised of Folic acid, Magnesium taurate, Niacin, Quercetin, Resveratrol, Vitamin B12, Vitamin B6, Vitamin D and Zinc.

2. A cardiovascular health-promoting nutraceutical composition, packaged as a multi-component liquid comprised of:
400 to 800 mcg Folic acid;
100 to 125 mg Magnesium taurate;
0. 5 to 50 mg Niacin;
100-500 mg Quercetin;
100 to 250 mg Resveratrol;
50 to 200 mcg Vitamin B12;
0.5 to 1 mg Vitamin B6;
1000 to 2000 IU Vitamin D3; and
5 to 25 mg Zinc

3. The composition of claim 2 further comprising 0.25 to 5 g L-arginine.

4. The composition of claim 2 further comprising 100 to 1000 mg Omega-3 fish oil.

5. The composition of claim 2 further comprising 200 to 1300 mg plant sterols.

6. The composition of claim 2 further comprising of 0.25 to 5 g of L-arginine, 100 to 1000 mg of Omega-3 fish oil, and 200 to 1300 mg plant sterols.

7. The composition of claim 6 further comprising 400 to 600 mg red yeast rice.

8. The composition of claim 6 further comprising a low-dose statin, such as 5-25 mg atorvastatin, as a polyceutical combination.

9. The composition of claim 6 further comprising 50-100 ubiquinol.

10. The composition of claim 6 further comprising 2-30 mg lycopene.

11. The composition of claim 6 further comprising 5-50 mg β-carotene.

12. The composition of claim 2 further comprising a solid.

13. The composition of claim 2 further comprising a pharmaceutical compound, as a polyceutical combination.

14. A cardiovascular health-promoting nutraceutical composition, prepared as a multi-component drink containing:
400 to 800 mcg Folic acid;
100 to 125 mg Magnesium taurate;
0.5 to 50 mg Niacin;
100-500 mg Quercetin;
100 to 250 mg Resveratrol;
50 to 200 mcg Vitamin B12;
0.5 to 1 mg Vitamin B6;
1000 to 2000 IU Vitamin D3; and
5 to 25 mg Zinc.

15. The composition of claim 14 further comprising 0.25 to 5 g L-arginine.

16. The composition of claim 14 further comprising 100 to 1000 mg Omega-3 fish oil.

17. The composition of claim 14 further comprising 200 to 1300 mg plant sterols.

18. The composition of claim 14 further comprising 0.25 to 5 g of L-arginine, 100 to 1000 mg of Omega-3 fish oil, and 200 to 1300 mg plant sterols.

19. The composition of claim 18 further comprising 400 to 600 mg red yeast rice.

20. The composition of claim 18 further comprising a low-dose statin, such as 5-25 mg atorvastatin.

21. The composition of claim 18 further comprising 50-100 mg ubiquinol.

22. The composition of claim 18 further comprising 2-30 mg lycopene.

23. The composition of claim 18 further comprising 5-50 mg β-carotene.

24. The composition of claim 6 further comprising carbonation.

25. The composition of claim 6 further added to any soft drinks.

26. The composition of claim 6 added to any tea.

27. The composition of claim 6 added to any coffee.

28. The composition of claim 6 in a form of a coffee creamer added to any soft drinks.

29. A polyceutical composition comprised of one or more nutraceutical compounds or compositions and one or more pharmaceutical compounds or compositions.

30. The polyceutical composition of claim 29 wherein the nutraceutical composition is comprised of one or more compounds which increase HDL or reduce LDL or promote cardiovascular function and the pharmaceutical compound is a statin medication.

31. The polyceutical composition of claim 28 wherein the nutraceutical composition is comprised of one or more components which boost metabolism and the pharmaceutical compound is an appetite suppressant.
